(19) United States
(12) Patent Application Publication
(10) Pub. No.: US 2011/0118522 A1
(43) Pub. Date: May 19, 2011

(54) OLEFIN FEED PURIFICATION PROCESS

(75) Inventors: Michael C. CLARK, Glen Mills, PA (US); Mark J. REICHENSPERGER, Billings, MO (US); Kevin J. BERNING, Plainfield, IL (US); Todd J. MILES, Ashburn, VA (US)

(73) Assignee: EXXONMOBIL RESEARCH AND ENGINEERING COMPANY, Annandale, NJ (US)

(21) Appl. No.: 12/899,049

(22) Filed: Oct. 6, 2010

Related U.S. Application Data
(60) Provisional application No. 61/281,162, filed on Nov. 13, 2009.

(51) Int. Cl. C07C 15/073; C07C 2/12; C07C 7/10; C07C 15/067

(52) U.S. Cl. 585/438; 585/717; 585/868

(57) ABSTRACT

A light olefin feed for an olefin conversion process is subjected to a water wash to remove water-soluble contaminants after which the water is separated from the olefin prior to the conversion reaction. The water used for the wash is free of boiler feedwater additives, especially basic nitrogenous additives, which adversely affect catalytic function.

### CROSS REFERENCE TO RELATED APPLICATIONS

This application relates to and claims priority to U.S. Provisional Patent Application No. 61/281,162, filed on Nov. 13, 2009.

### FIELD OF THE INVENTION

This invention relates to a method of purifying the olefin feeds used in an olefin conversion process and to the conversion process with the feed purification.

### BACKGROUND OF THE INVENTION

US Patent Application Publication No. 2006/0194999, entitled “Gasoline Production by Olefin Polymerization” describes a process for the production of high quality hydrocarbon fuels in the gasoline boiling range by the polymerization (actually, oligomerization although the term “polymerization” is often used in reference to the process) of low molecular weight olefins, principally olefins from FCC fuel gas, mainly ethylene and propylene and possibly butene.

US Patent Application Publication No. 2006/0194998, entitled “Process for Making High Octane Gasoline with Reduced Benzene Content” describes a process for the production of high quality hydrocarbon fuels in the gasoline boiling range by the alkylation of reformates and other light aromatic refinery streams with low molecular weight olefins. This reformate alkylation process may be combined with the olefin oligomerization process as described in U.S. Pat. No. 7,525,002 entitled “Gasoline Production by Olefin Polymerization with Aromatics Alkylation” where the oligomerization is combined in unit with the reformate alkylation. A variant of the process is described in U.S. Pat. No. 7,476,774, entitled “Liquid Phase Aromatics Alkylation Process” in which the alkylation is carried out in the liquid phase; another variant in which the alkylation is carried out in the vapor phase is described in U.S. Pat. No. 7,498,474, entitled “Vapor Phase Aromatics Alkylation Process”. Operation of the process with high levels of benzene is described in U.S. patent application Ser. No. 12/720,345, entitled “Process for Making High Octane Gasoline with Reduced Benzene Content by Benzene Alkylation at High Benzene Conversion” the disclosure of which is hereby incorporated in its entirety herein specifically by reference.

In conventional olefin oligomerization processes the catalyst is a solid phosphoric acid catalyst made by sorbing phosphoric acid on kieselguhr but a significant improvement in the process is achieved as described in US Patent Application Publication No. 2006/0194999 by using a zeolite catalyst preferably of the MWW family. Both the conventional SPA catalyst and the zeolite catalysts are prone to poisoning by trace quantities (ppm levels) of contaminants such as acetonitrile (ACN), amines, sodium which may be present as carryover from the FCC or which are introduced during caustic and water wash steps used to purify the feed. The zeolite catalysts are more robust than the SPA catalyst but they nevertheless are sensitive to organic compounds with basic nitrogen as well as sulfur-containing organics. It is therefore preferred to remove these materials prior to entering the oligomerization unit if extended catalyst life is to be expected, both in the olefin oligomerization process and the reformate alkylation process which are both subsumed and referred to in this application as olefin conversion processes.

Scrubbing with contaminant removal washes such as caustic, MEA (monoethanolamine) or other amines or aqueous wash liquids will normally reduce the sulfur level to an acceptable level of about 10-20 ppmw and the nitrogen to trace levels at which it can be readily tolerated. Although activity benefits are achieved by the use of low or very low water levels in the feed, the zeolite catalysts are not otherwise unduly sensitive to water, making it less necessary to control water entering the reactor than it is with SPA units. Unlike SPA, the zeolite catalyst does not require the presence of water in order to maintain activity and therefore the feed may be dried before entering the unit, for example, to below 200 ppmw water or lower, e.g. below 50 or even 20 ppmw. In conventional SPA units, the water content typically needs to be held between 300 to 500 ppmw at conventional operating temperatures for adequate activity while, at the same time, retaining catalyst integrity. The zeolite catalysts, however, may readily tolerate higher levels of water up to about 1,000 ppmw water although levels above about 800 ppmw may reduce activity, depending on temperature. Thus, with converted units, the olefin feed may contain from 300 or 500 to 1,000 ppmw water, although 300-800 ppmw should be regarded as a workable range for activity with existing feed treatment equipment.

The use of a guard bed prior to the olefin conversion reactor may be desirable since the refinery feeds customarily routed to conversion units (as distinct from petrochemical unit feeds which are invariably high purity feeds for which no guard bed is required) may have a contaminant content, especially of polar catalyst poisons, such as the polar organic nitrogen and organic sulfur compounds, which is too high for extended catalyst life. The use of a cheaper catalyst in the guard bed reactors which can be readily regenerated in swing cycle operation or, alternatively disposed of on a once-through basis, is normally viewed as desirable in ensuring extended cycle duration for the active zeolite catalyst.

While a water wash is effective to remove basic nitrogenous contaminants such as amines, large quantities may be necessary to achieve adequate feed purity presenting not only a problem in the wash units themselves but also in the disposal of the contaminated water after the wash. Another problem is that excess levels of water in the feed may depress the activity of zeolite catalysts or, in the case of SPA catalysts, result in disintegration and complete inactivation of the catalyst. For these reasons, feed purification is an important factor in the process regardless of the catalyst actually used.

### SUMMARY OF THE INVENTION

Conventionally, the water used for feed washing in petroleum refineries is boiler feed water which contains various additives: anti-corrosion agents and oxygen scavengers are typically used to inhibit corrosion in boilers and other equipment, anti-foaming agents to inhibit foaming, scale inhibitors in hard water areas, pH adjusters, sludge conditioners. Anti-corrosion agents are typically nitrogenous basic species while oxygen scavengers may typically be tannin or sulfite based. The additives used for pH adjustment are often alkalinity agents to offset the effect of acidity in the feed water, for example caustic or caustic/polymer combinations. Anti-foaming additives are typically low molecular weight, water-soluble polymers such as the ethers of ethylene oxide/propylene oxide polyglycols. Sludge conditioners may be based on phosphates or polymers or a combination of both.

We have found that commonly used boiler feed water additives have been found to have a deleterious effect on the zeolite catalysts that may be used in the olefin conversion processes; in particular, nitrogen-based basic compounds often used as anti-corrosion agents bind to the acid sites of MWW and other zeolites used in the olefin conversion and deactivate it quickly. Although the poisoning effect of the zeolite may be temporary and capable of being reversed by hydrogen reactivation at elevated temperature, the reactivation procedure requires production to be stopped with its consequent downtime and economic losses.

According to the present invention, an olefin conversion process in which a light (C₂-C₆) olefin feed stream is converted to a higher boiling product in the gasoline boiling range by polymerization or alkylation of a light aromatic compound (monocyclic) uses an intermediate pore size zeolite conversion catalyst to effect the conversion. The olefin feed stream is washed with water prior to the conversion over the zeolite catalyst using water that is free of boiler feedwater additives, especially basic nitrogenous compounds.

### DETAILED DESCRIPTION

### Olefin Conversion Processes

The present olefin purification process may be applied to purifying the light olefin feeds to any process in which the feeds are to be utilized, typically in catalytic processes using a solid catalyst, especially a solid zeolite catalyst, The present purification process is particularly applicable to the purification of light olefin feeds which are to be used in oligomerization to higher hydrocarbons, especially fractions boiling in the gasoline boiling range, in the alkylation of reformates and other aromatic feeds for example, in the production of gasoline boiling range fractions with reduced benzene content, the production of cumene by the alkylation of benzene, the production of ethylbenzene by the ethylation of benzene with ethylene, the production of sec-butyl benzene by the alkylation of benzene with n-butene. Processes of this kind are described, for example in the following: U.S. Patent Publication No. 2006/0194999 (Olefin Oligomerization); U.S. Patent Publication No. 2006/0194998 (Reformate Alkylation); U.S. Pat. No. 7,498,474 (Vapor Phase Reformate Alkylation); U.S. Pat. No. 7,476,774 (Liquid Phase Reformate Alkylation); U.S. Pat. No. 7,525,002 (Olefin Polymerization with Aromatics Alkylation); U.S. patent application Ser. No. 12/720,345 (Reformate Alkylation with High Benzene Conversion), U.S. Pat. No. 6,888,037 (Cumene Production) U.S. Pat. No. 5,493,065 (Ethylbenzene Production) and U.S. Pat. No. 7,671,248, (Production of sec-butyl benzene), to which reference is made for descriptions of such processes, these descriptions being specifically incorporated herein in their entirety by reference here. These patents and publications cited here are purely exemplary and other processes for these and other reactions using light olefin feeds with these and other co-reactants and other catalysts are well-known; the application of the present olefin purification technique is generally applicable to all such processes.

### Olefin Feed

The light olefins used as the feed in the present olefin conversion processes are normally obtained by the catalytic cracking of petroleum feedstocks to produce gasoline as the major product. The catalytic cracking process, usually in the form of fluid catalytic cracking (FCC) produces large quantities of light olefins as well as olefinic gasolines and by-products such as cycle oil which are themselves subject to further refining operations. The olefins which are primarily useful in the present process are the lighter olefins from ethylene up to butene (C₂-C₄); although the heavier C₅+ olefins may also be included in the processing, they can generally be incorporated directly into the gasoline product where they provide a valuable contribution to octane. The oligomerization and reformate alkylation processes will operate readily not only with butene and propylene but also with ethylene and thus provide a valuable route for the conversion of this cracking by-product to desired gasoline products. For this reason as well as their ready availability in large quantities in a refinery, mixed olefin streams such a FCC Off-Gas streams (typically containing ethylene, propylene and butenes) may be used. Oligomerization of the C₃ and C₄ olefin fractions from the cracking process provides a direct route to the branch chain C₆, C₇ and C₈ products which are so highly desirable in gasoline from the view point of boiling point and octane while reformate alkylation with propylene and butene provides a route to the high octane aromatics mostly in the desirable C₉ and C₁₀ boiling range. Besides the FCC unit, the mixed olefin streams may be obtained from other process units including cokers, visbreakers and thermal crackers. The presence of diolefins which may be found in certain refinery streams such as those from thermal cracking is not desirable in view of their tendency to form high molecular weight polymerization products which indicates that their removal in a diolefin saturation unit is preferred.

The compositions of two typical FCC gas streams is given below in Tables 1 and 2, Table 1 showing a light FCC gas stream and Table 2 a stream from which the ethylene has been removed in the gas plant for use in the refinery fuel system.

TABLE 1
FCC Light Gas Stream

| Component | Wt. Pct. | Mol. Pct. |
|---|---|---|
| Ethane | 3.3 | 5.1 |
| Ethylene | 0.7 | 1.2 |
| Propane | 14.5 | 15.3 |
| Propylene | 42.5 | 46.8 |
| Iso-butane | 12.9 | 10.3 |
| n-Butane | 3.3 | 2.6 |
| Butenes | 22.1 | 18.32 |
| Pentanes | 0.7 | 0.4 |

TABLE 2
C₃-C₄ FCC Gas Stream

| Component | Wt. Pct. |
|---|---|
| 1-Propene | 18.7 |
| Propane | 18.1 |
| Isobutane | 19.7 |
| 2-Me-1-propene | 2.1 |
| 1-Butene | 8.1 |
| n-Butane | 15.1 |
| Trans-2-Butene | 8.7 |
| Cis-2-butene | 6.5 |
| Isopentane | 1.5 |
| C3 Olefins | 18.7 |
| C4 Olefins | 25.6 |
| Total Olefins | 44.3 |

### Aromatic Feed

In the reformate alkylation process, a light aromatic feed containing benzene is alkylated with the light olefin feed. This stream may contain other single ring aromatic compounds including alkylaromatics such as toluene, ethylbenzene, propylbenzene (cumene) and the xylenes. In refineries with associated petrochemical capability, these alkylaromatics will normally be removed for higher value use as chemicals or, alternatively, may be sold separately for such uses. Since they are already considered less toxic than benzene, there is no environmental requirement for their inclusion in the aromatic feed stream but, equally, there is no prejudice against their presence unless conditions lead to the generation of higher alkylaromatics which fall outside the gasoline range or which are undesirable in gasoline, for example, tetra-isopropylbenzene. The amount of benzene in this stream is governed mainly by its source and processing history but in most cases will typically contain at least about 3 vol. % benzene, although a minimum of 12 vol. % is more typical, more specifically about 20 vol. % to 40 vol. % benzene. Normally, the main source of this stream will be a stream from the reformer which is a ready source of light aromatics. Reformate streams may be full range reformates, light cut reformates, heavy reformates or heart cut reformates. These fractions typically contain smaller amounts of lighter hydrocarbons, typically less than about 10% C₅ and lower hydrocarbons and small amounts of heavier hydrocarbons, typically less than about 15% C₇+ hydrocarbons. These reformate feeds usually contain very low amounts of sulfur as, usually, they have been subjected to desulfurization prior to reforming so that the resulting gasoline product formed in the present process contains an acceptably low level of sulfur for compliance with current sulfur specifications.

Reformate streams will typically come from a fixed bed, swing bed or moving bed reformer. The most useful reformate fraction is a heart-cut reformate. This is preferably reformate having a narrow boiling range, i.e. a C₆ or C₆/C₇ fraction. This fraction is a complex mixture of hydrocarbons recovered as the overhead of a dehexanizer column downstream from a depentanizer column. The composition will vary over a range depending upon a number of factors including the severity of operation in the reformer and the composition of the reformer feed. These streams will usually have the C₅, C₄ and lower hydrocarbons removed in the depentanizer and debutanizer. Therefore, usually, the heart-cut reformate will contain at least 70 wt. % C₆ hydrocarbons, and preferably at least 90 wt. % C₆ hydrocarbons.

Other sources of aromatic, benzene-rich feeds include a light FCC naphtha, coker naphtha or pyrolysis gasoline but such other sources of aromatics will be less important or significant in normal refinery operation.

By boiling range, these benzene-rich fractions can normally be characterized by an end boiling point of about 120° C. (250° F.), and preferably no higher than about 110° C. (230° F.). Preferably, the boiling range falls between 40° and 100° C. (100° F. and 212° F.), and more preferably between the range of 65° to 95° C. (150° F. to 200° F.) and even more preferably within the range of 70° to 95° C. (160° F. to 200° F.).

The compositions of two typical heart cut reformate streams are given in Tables 3 and 4 below. The reformate shown in Table 4 is a relatively more paraffinic cut but one which nevertheless contains more benzene than the cut of Table 3, making it a very suitable substrate for the present alkylation process.

TABLE 3
C6-C7 Heart Cut Reformate

| | |
|---|---|
| RON | 82.6 |
| MON | 77.3 |
| Composition, wt. pct. | |
| i-C₅ | 0.9 |
| n-C₅ | 1.3 |
| C₅ naphthenes | 1.5 |
| i-C₆ | 22.6 |
| n-C₆ | 11.2 |
| C₆ naphthenes | 1.1 |
| Benzene | 32.0 |
| i-C₇ | 8.4 |
| n-C₇ | 2.1 |
| C₇ naphthenes | 0.4 |
| Toluene | 17.7 |
| i-C₈ | 0.4 |
| n-C₈ | 0.0 |
| C₈ aromatics | 0.4 |

TABLE 4
Paraffinic C6-C7 Heart Cut Reformate

| | |
|---|---|
| RON | 78.5 |
| MON | 74.0 |
| Composition, wt. pct. | |
| i-C₅ | 1.0 |
| n-C₅ | 1.6 |
| C₅ naphthenes | 1.8 |
| i-C₆ | 28.6 |
| n-C₆ | 14.4 |
| C₆ naphthenes | 1.4 |
| Benzene | 39.3 |
| i-C₇ | 8.5 |
| n-C₇ | 0.9 |
| C₇ naphthenes | 0.3 |
| Toluene | 2.3 |

Reformate streams will come from a fixed bed, swing bed or moving bed reformer. The most useful reformate fraction is a heart-cut reformate. This is preferably reformate having a narrow boiling range, i.e. a C₆ or C₆/C₇ fraction. This fraction is a complex mixture of hydrocarbons recovered as the overhead of a dehexanizer column downstream from a depentanizer column. The composition will vary over a range, depending upon a number of factors including the severity of operation in the reformer and the composition of the reformer feed. These streams will usually have the C₅, C₄ and lower hydrocarbons removed in the depentanizer and debutanizer. Therefore, usually, the heart-cut reformate may contain at least 70 wt. % C₆ hydrocarbons (aromatic and non-aromatic), and preferably at least 90 wt. % C₆ hydrocarbons.

Other sources of aromatic, benzene-rich feeds include a light FCC naphtha, coker naphtha or pyrolysis gasoline but such other sources of aromatics will be less important or significant in normal refinery operation.

### Olefin Conversion Catalyst

The present feed purification technique is generally applicable with olefin utilization processes using a solid, acidic catalyst which is preferably a molecular sieve catalyst, although an acidic amorphous catalyst such as SPA (solid phosphoric acid) will also function although less effectively than the preferred zeolite catalysts. Zeolites with 10-membered ring systems are preferred in these reactions, especially zeolites of the MEL, MFI, MFS, MTT, MTW, NU-87, MWW and TON structural types having the requisite degree of acidic functionality.

Zeolites of the MWW family are preferred for many of these reactions including olefin oligomerization, reformate alkylation, and for cumene and ethylbenzene production This family is currently known to include a number of zeolitic materials such as PSH-3 (described in U.S. Pat. No. 4,439,405), MCM-22 (described in U.S. Pat. No. 4,954,325), MCM-36 (described in U.S. Pat. No. 5,250,277), MCM 49 (described in U.S. Pat. No. 5,236,575), MCM 56 (described in U.S. Pat. No. 5,362,697), SSZ 25 (described in U.S. Pat. No. 4,826,667), ERB-1 (described in European Patent No. EP 0293032), ITQ-1 (described in U.S. Pat. No. 6,077,498), ITQ-2 (described in WO 97/17290), UZM-8 (described in U.S. Pat. No. 6,756,030). Of these, the four significant members for use as olefin oligomerization catalysts are MCM-22, MCM-36, MCM-49, and MCM-56 with preference given to MCM-22 and MCM-49. It has been found that the MCM-22 or MCM-49 catalysts may be either used fresh, that is, not having been previously used as a catalyst or alternatively, regenerated catalyst may be used. Regenerated catalyst may be used after it has been used in any of the catalytic processes for which it is suitable, including the present process in which the catalyst has shown itself to remain active even after multiple regenerations. It may also be possible to use MWW catalysts which have previously been used in other commercial processes and for which they are no longer acceptable, for example, catalyst which has previously been used for the production of aromatics such as ethylbenzene or cumene, normally using reactions such as alkylation and transalkylation, as described in U.S. Patent Application Publication No. 2006/0194998. A full description of the MWW zeolites which are the optimal zeolite catalysts is given in US Patent Application Publication No. 2006/0194998 and US Patent Application Publication No. 2006/0194999, to which reference is made for a full description of these catalysts and the manner in which they may be used in the conversion of the light olefin feeds by oligomerization and light aromatic alkylation.

### Olefin Conversion Conditions

The olefin conversion reaction is carried out at the appropriate conditions for the reaction and its desired products. The olefin oligomerization reaction, for example, is carried out under conditions appropriate to the equipment and catalyst in use. For oligomerization using a MWW zeolite catalyst, suitable reaction conditions will be as described in as US Patent Application Publication No. 2006/0194999. Briefly, the process may be operated at low to moderate temperatures and pressures. In general, the temperature will be from about 120° to 250° C. (about 250° to 480° F.) and in most cases between 150° and 250° C. (about 300°-480° C.). Temperatures of 170° to 205° C. (about 340° to 400° F.) will normally be found optimum for feeds comprising butene while higher temperatures will normally be appropriate for feeds with significant amounts of propene. Pressures may be those appropriate to the type of unit so that pressures up to about 7500 kPag (about 1100 psig) will be typical but normally lower pressures will be sufficient, for example, below about 7,000 Kpag (about 1,000 psig) and lower pressure operation may be readily utilized, e.g. up to 3500 kPag (about 500 psig). Pressures of this order are consistent with those preferred for the water wash as noted below, therefore enabling the entire unit to be operated without de- or re-pressurization at any point. Ethylene, again, will require higher temperature operation to ensure that the products remain in the gasoline boiling range. Space velocity may be quite high, for example, up to 50 WHSV (hr⁻¹) but more usually in the range of 5 to 30 WHSV. Appropriate adjustment of the process conditions will enable co-condensation products to be produced when ethylene, normally less reactive than its immediate homologs, is included in the feed. Other olefin utilization reactions will be carried out at tier own respective appropriate conditions as described, for example, in the publications referred to above. Reference is made to US Patent Application Publication No. 2006/0194999 for a more detailed description of suitable reaction conditions for this olefin oligomerization process.

Reaction conditions appropriate to the reformate alkylation process include temperatures from about 120° to 350° C. (about 250 to 660° F.) and in most cases between 150° and 250° C. (about 300 to 480° F.). Temperatures of 170° to 180° C. (340° to 355° F.) will normally be found optimum for feeds comprising butene while higher temperatures will normally be appropriate for feeds with significant amounts of propene. Ethylene will require higher temperature operation to ensure satisfactory ethylene conversion. Pressures will normally be dependent on unit constraints but usually will not exceed about 10,000 kPag (about 1450 psig) with low to moderate pressures, normally not above 7,000 kPag (about 1,000 psig) being favored from equipment and operating considerations although higher pressures are not unfavorable in view of the volume change in the reaction; in most cases, the pressure will be in the range of 2000 to 5500 kPag (about 290 to 800 psig) in order to make use of existing equipment. Space velocities can be quite high, giving good catalyst utilization. Space velocities are normally in the range of 0.1 to 5 hr⁻¹ WHSV for the olefin feed, in most cases, 0.5 to 1 hr⁻¹ WHSV. Optimum conditions may be determined empirically, depending on feed composition, catalyst aging and unit constraints.

Two factors affecting choice of temperature will be the feed composition and the presence of impurities, principally in the olefin feed stream. As noted above, ethylene is less reactive than propylene and for this reason, ethylene containing feeds will require higher temperatures than feeds from which this component is absent, assuming of course that high olefin conversion is desired. From this point of view, reaction temperatures at the higher end of the range, i.e. above 180° C. or higher, e.g. 200° or 220° C. or higher, will be preferred for ethylene-containing feeds. Sulfur will commonly be present in the olefin feeds from the FCC unit in the form of various sulfur-containing compounds e.g. mercaptans, and since sulfur acts as a catalyst poison at relatively low reaction temperatures, typically about 120° C., but has relatively little effect at higher temperatures about 180° C. or higher, e.g. 200° C., 220° C., the potential for sulfur compounds being present may dictate a preferred temperature regime above about 150° C., with temperatures above 180° C. or higher being preferred, e.g. 200° or 220° C. or higher. Typically, the sulfur content will be above 1 ppmw sulfur and in most cases above 5 ppmw sulfur; it has been found that with a reaction temperature above about 180-220° C., sulfur levels of 10 ppmw can be tolerated with no catalyst aging, indicating that sulfur levels of 10 ppmw and higher can be accepted in normal operation.

High benzene conversion may be attained by operation under the conditions described in U.S. patent application Ser. No. 12/720,345. These conditions include maintaining the aromatic stream in the liquid phase with the pressure maintained at a value high enough to ensure subcritical operation, typically at values above about 4000 kPag (about 580 psig) although pressures as low as about 2500 kPag (about 360 psig) may be operable depending on the feedstream composition and the temperature. Minimum temperatures are generally in the range of 175-200° C. (347-392° F.), more usually at least 220° C. (428° F.); the maximum will not normally exceed 300° C. (572° F.) with a maximum of 250° C. (482° F.) being normally preferred. Operation with at least a partial liquid phase is most desired as determined by composition, pressure and temperature of each specific unit. Control of the exotherm may be assisted by the staged injection of the olefin in at least two beds; interbed heat removal and/or recirculation of cooled reactor product. Once-through operation promote higher benzene conversion together with a reduction of product endpoint (by about 25° C.) and a reduction in the volume of product with end point above the mogas range; a reduction of approximately 50% in the volume of product boiling above the mogas endpoint specification is achievable. Single pass operation with an propylene-containing olefinic feed stream (at least 50 weight percent propylene) is a factor conducive to conversion of at least 70 weight percent of the benzene in the reformate feed to alkylbenzenes and is specifically preferred for benzene conversion above 90-95%. A more detailed description of the appropriate conditions for high benzene conversion can be taken from U.S. patent application Ser. No. 12/720,345, to which reference is made for a description of such conditions.

### Water Wash

The light olefin feed is typically first subjected to an amine wash to remove H2S and other light S compounds. The light olefin feed is next typically subjected to a wash with an aqueous wash liquid which is typically caustic. Caustic wash is especially effective to remove mercaptans and other sulfur impurities. The light olefin feed is finally typically subjected to a water wash which is effective for nitrogenous and other water-soluble bases. The ratio of water to olefin is typically in the range of 1:1 to 10:1 by weight. Wash unit design will be conventional with the objective of ensuring good contact between the olefin feed and the water with various types of contactor applicable, for example, scrubbers, countercurrent towers. A once-through water wash is preferred to operation with recycled water and pH control of the water is most desirable to ensure efficient removal of the N-based species. The pH should desirably be held in the range of pH: 5-8, preferably 5.5-7, and more preferably 5.5-6.5 for this purpose. During this step and the following coalescence step, the conditions should be chosen so as to maintain the olefin stream in the liquid phase since this will favor removal of the contaminant species. The preferred temperatures for the feed and the wash water below about 40° C. and preferably below 35° C., since water tends to be more soluble in the feed at higher temperatures and so tends to dissolve in the feed and be less amenable to separation by coalescence and, remaining in the feed will take with it the water soluble contaminants such as acetonitrile and nitrogenous bases. Thus, operation of the water wash and the subsequent coalescence not only reduces the water content of the feed, in itself desirable, but also reduces the level of impurities in the feed. Operation at temperatures below about 25 or 20° C. can be desirable from this point of view. At the preferred temperatures, the olefins will be in the liquid phase at pressures of about 2100-4200 kPa (approx. 300-600 psia.)

A supplemental wash step may follow the initial wash to help dissolve soluble poisons prior to separation of the water. The amount of water used in this step relative to the feed is typically from about 0.5:1 to 2:1 by weight.

It has been surprisingly discovered that the treatment of olefin feed with water contaminated with undesirable compounds can actually produce an olefin feedstock less suitable for processing than an untreated feed. N-based catalyst poisons can migrate from boiler feed water, for example, into the olefin feed during contacting thereby increasing the N-content of the olefin feed. In the event that contaminated wash water is used, subsequent treatment of the olefin feed with a molecular sieve for example could be sufficient to remove the impurities from the olefin feed. This pretreatment comes, however, at increased cost relative to the use of the water which contains no significant amounts of deleterious feedwater additives.

According to the present invention, therefore, the water wash is carried out using water which is free, that is, contains no significant amounts, of boiler feedwater additives. In particular, nitrogenous basic species which bind to the acidic sites on the zeolite catalysts are to be absent. Water of this quality can be found by using sources such as artesian spring water, purified river water, desalinated water, e.g. by distillation or reverse osmosis, de-ionized water, and drinking water. The wash is preferably carried out using water that contains less than 10 ppmw, desirably less than 5 ppmw, total nitrogen and less than 5 ppmw ammonia.

River water may be purified by filtration, e.g. by slow sand filter or lava filter. These types of filter rely on biological treatment processes for their action rather than physical filtration. The filters are constructed with graded layers of sand with the coarsest sand, along with some gravel, at the bottom and finest sand at the top. The treated water is removed from the base of the filter. Other extractive purification techniques such as reverse osmosis, distillation, ultrafiltration, ion exchange, electro de-ionization, may also be used but additive purification, e.g. by the addition of phosphates to inhibit lead solvency, is to be avoided in view of the objective of providing a water source without additives of the type used in boiler feed water or, for that matter, of other components likely to adversely affect the catalytic action of the zeolite catalysts in the conversion process.

The removal of basic nitrogenous components from the water is important since these have a serious adverse effect on catalyst action. A water wash is effective to remove basic nitrogenous contaminants such as amines and can be carried out in an otherwise conventional manner. After the wash is completed, the water is desirably removed to result in a dry feed since excess levels of water in the feed may depress the activity of zeolite catalysts or, in the case of SPA catalysts, result in disintegration and complete inactivation of the catalyst. Absolute dryness is not, however, required with the zeolite catalysts used in the conversion.

Removal of water from the olefin feed stream may be carried out conventionally, e.g. by the use of dryers such as silica gel or molecular sieve dryers with zeolite 4A being especially effective. The dryers can be operated in swing bed mode with the off-stream dryer being regenerated while the on-stream dryer is in operation.

Another drying method that may be used is coalescence, as described in U.S. patent application Ser. No. 12/718,700, to which reference is made for a description, now incorporated, of olefin feed stream drying by coalescence filters. Yet another alternative is the use of a Superabsorbent Polymer (SAP). Superabsorbent polymers (also called slush powder) are polymers that can absorb and retain extremely large amounts of a liquid relative to the mass of the polymer used. Superabsorbing polymers which take up water, classified as hydrogels, absorb aqueous solutions through hydrogen bonding with the water molecule. The use of superabsorbent polymers in this way is also described in U.S. patent application Ser. No. 12/718,700, to which reference is made for a description, now incorporated, of olefin feed stream drying in this way.

### Example 1

Reformate alkylation was carried out in a pilot unit using a refinery reformate having the composition given in Table 5 below.

TABLE 5
Reformate Feed Composition

| | Wt. Pct. |
|---|---|
| Benzene | 6.59 |
| Toluene | 25.6 |
| Ethylbenzene | 2.47 |
| Paraxylene | 2.46 |
| Metaxylene | 5.89 |
| Orthoxylene | 2.93 |
| Cumene | 0.12 |
| 1,4-Ethyltoluene | 1.36 |
| 1,2-Ethyltoluene | 0.32 |
| 1,3,5-Trimethylbenzene | 0.42 |
| 1,2,4-1,2,4-Trimethylbenzene | 1.21 |
| 1,2,3-Trimethylbenzene | 0.2 |
| N-Propylbenzene | 0.37 |
| Mixed C10's | 0.6 |
| Nonaroms | 49.4 |

This reformate was alkylated using a refinery LPG feed with the composition (mole fraction); C3=0.15, C3=0.20, iC4=0.15, C4==0.24, nC4=0.24)) which had been treated with amine, caustic and water with no anti-corrosion agents added. The alkylation was carried out over an MCM-22 catalyst at 204° C. (400° F.), 7000 kPag (1000 psig) at a space velocity of 1 hr.−1 LHSV on the LPG, 1.4 hr.−1 LHSV on the reformate. Benzene conversion remained stable at a nominal 80 vol. pct. over a period of 13 days.

### Example 2

Example 1 was repeated but using; first, commercially available specialty propylene (99 wt. % propylene without N-based species), and Second a refinery LPG of different composition which was washed with water which contained basic nitrogenous anti-corrosion agents two days after start of run.

TABLE 6
Analysis of water in LPG pretreatment

| | | NH3 | Total N |
|---|---|---|---|
| | pH | (ppm) | (ppm) |
| Condensate (Water pre-anti | 9.0 | 0 | 2 |
| corrosion agent addition) | | | |
| Water Wash (Condensate post | 11.3 | 75 | 37 |
| anti-corrosion agent addition) | | | |
| Spent Water Wash (Spent water | 12.2 | 50 | 148 |
| from LPG wash) | | | |

The reformate was alkylated at an initial temperature of 204° C. (400° F.) and 7000 kPag (1000 psig) at the same space velocities relative to the two feeds as in Example 1. For the first two days of the run, the commercially available specialty propylene (99 wt. % propylene) was the feed source with no N-based species present. This feedstock produced stable catalyst activity. After two days, the feed was changed to a refinery LPG stream which had been washed with water containing a nitrogenous, basic anti-corrosion agent, resulting in rapid catalyst deactivation.

1. A process for purifying a light (C₂-C₆) olefin feed for an olefin conversion process which comprises subjecting the feed to a wash with water free of boiler feedwater additives and separating the washed olefin feed from the wash water prior to conversion.

2. A process according to claim 1 in which the water wash is carried out at a temperature below 40° C. and at a pressure of 2100-4200 kPa with the olefin feed in the liquid phase.

3. A process according to claim 2 in which the water wash is carried out at a temperature below 35° C.

4. A process according to claim 1 in which the light olefin feed comprises olefins from ethylene up to butene.

5. A process according to claim 4 in which the light olefin feed comprises propylene, propylene and ethylene or propylene and butene.

6. A process according to claim 1 in which the wash water contains less than 10 ppmw total nitrogen and less than 5 ppmw ammonia.

7. A process according to claim 6 in which the wash water contains less than 5 ppmw total nitrogen and less than 5 ppmw ammonia.

8. In a light olefin conversion process in which a light (C₂-C₆) olefin feed is converted to higher boiling reaction products in the gasoline boiling range in a reaction over a solid zeolite catalyst, the improvement comprising purifying the light olefin feed by subjecting the feed to a wash with water free of boiler feedwater additives and separating the washed olefin feed from the wash water prior to conversion.

9. A process according to claim 8 in which the water wash is carried out at a temperature below 40° C. and at a pressure of 2100-4200 kPa with the olefin feed in the liquid phase.

10. A process according to claim 9 in which the water wash is carried out at a temperature below 35° C.

11. A process according to claim 8 in which the light olefin feed comprises olefins from ethylene up to butene.

12. A process according to claim 11 in which the light olefin feed comprises propylene, propylene and ethylene or propylene and butene.

13. A process according to claim 8 in which the wash water contains less than 10 ppmw total nitrogen and less than 5 ppmw ammonia.

14. A process according to claim 13 in which the wash water contains less than 5 ppmw total nitrogen and less than 5 ppmw ammonia.

15. A process according to claim 8 in which the olefin conversion process is olefin oligomerization.

16. A process according to claim 8 in which the olefin conversion process is reformate alkylation.

17. A process according to claim 8 in which the olefin conversion process is alkylation of a benzene feed to make cumene or ethylbenzene.
